(19) United States
(12) Patent Application Publication
(10) Pub. No.: US 2024/0287436 A1
(43) Pub. Date: Aug. 29, 2024

(54) RAMAN SPECTROSCOPY METHOD AND SYSTEM FOR MONITORING INVASIVE SPECIES IN ALGAL BIOREACTORS

(71) Applicant: The State of Israel, Ministry of Agriculture & Rural Development, Agricultural Research, Rishon Lezion (IL)

(72) Inventors: Orr SHAPIRO, Rehovot (IL); Ze'ev SCHMILOVITCH, Yehud-Monoson (IL); Timea IGNAT, Midreshet Ben Gurion (IL); Olubunmi ADEJIMI, Rishon Lezion (IL)

(21) Appl. No.: 18/572,258

(22) PCT Filed: Jun. 20, 2022
(86) PCT No.: PCT/IB2022/055712

§ 371 Date: Dec. 20, 2023

Related U.S. Application Data
(60) Provisional application No. 63/212,672, filed on Jun. 20, 2021.

(51) Int. Cl. C12M 1/34; G01N 21/65

(52) U.S. Cl. CPC C12M 41/36; G01N 21/65; G01N 2201/06113; G01N 2201/08; G01N 2201/127

(57) ABSTRACT

A method and system for monitoring invasive species in an algal bioreactor utilizes a low resolution Raman spectrometer (LRRS) system and signal processing methods based upon Support Vector Machine (SVM) models. A spectrum preprocessing algorithm transforms LRRS spectra into normalized spectral data vectors. An ex-situ method is used to analyze a calibration set of known samples of a suspension containing a desired biomass, such as spirolina, together with various concentrations of invasive algal or cyanobacterial species. The ex-situ method generates SVM models and their associated SVM support vectors for classification modeling. An in-situ method uses the SVM models to provide an output vector of probability values corresponding to the presence of invasive species in an unknown sample, and optionally to provide an additional output vector of probability values corresponding to concentrations of the invasive species in the unknown sample.

### CROSS-REFERENCE TO RELATED APPLICATIONS

This application is related to and claims priority from commonly owned U.S. Provisional Patent Application No. 63/212,672, entitled “Raman Spectroscopy System and Method for Monitoring Invasive Species in Algal Bioreactors”, filed on Jun. 20, 2021, the disclosure of which is incorporated by reference in its entirety herein.

### TECHNICAL FIELD

The present invention relates to low-resolution Raman spectroscopy (LRRS) systems and chemometric methods for detecting and monitoring invasive species in algal bioreactors.

### BACKGROUND OF THE INVENTION

Bioreactors are used to cultivate cyanobacterial and algal species, such as Spirulina, in controlled environments such as open or semi-enclosed raceways or photo-bioreactors. Such systems are often prone to contamination by invasive algal or cyanobacterial species. Some invading cyanobacteria and microalgae not only degrade the quality of the product for human consumption, but may also produce secondary metabolites that are toxic and possibly carcinogenic as well. To protect consumers, the World Health Organization (WHO) and the regulatory agencies of many countries have established standards placing severe limits on the maximum daily intake of such toxins. These limits can be converted into limits on the maximum allowable concentration of harmful contaminants inside a bioreactor or in the final product.

Currently available methods for the detection of invasive microalgal species and their toxins include optical microscopy, DNA-based methods, and enzyme-linked immunosorbent assay (ELISA). However, these methods are either not sensitive enough, or are too expensive or time-consuming to be economically feasible for monitoring bioreactors. For example, in images collected using optical microscopy, it is difficult to detect the presence of Microcystis aeruginosa cyanobacterial contamination, having a concentration of 10⁵ cells/ml (cells/milliliter), in a Spirulina sample containing 10⁸ cells/ml of the species Arthrospira platensis.

Low-resolution Raman Spectroscopy (LRRS) is a low-cost and sensitive method for detecting chemical constituents in a bulk sample. In LRRS, a sample is illuminated with a powerful monochromatic laser, and inelastic photon scattering produces a shift in the scattered wavelengths which is characteristic of the chemical bonds found in the chemical compounds within the sample. In analyzing aqueous solutions, Raman spectroscopy has the advantage of having a low sensitivity to water and a relatively high sensitivity and specificity for other components.

For example, a technical paper by Z. Schmilovitch et al., entitled “Detection of Bacteria with Low-Resolution Raman Spectroscopy”, published in Transactions of the American Society of Agrigultural Engineers, September 2005, pp. 1843-1850, proposes the use of LRRS as a rapid and reliable tool for on-site product safety assessment. The paper evaluates the sensitivity of LRRS to detect the presence of plant bacteria in a dilute suspension containing a mixture of two different bacteria, and to provide an estimate of their concentrations.

Machine learning chemometric algorithms involve the use of mathematical and statistical methods to analyze spectral data such as LRRS together with chemical variables. Such methods include, for example, Principal Components Analysis (PCA), Artificial Neural Network (ANN), Partial Least Squares Discriminant Analysis (PLSDA), Support Vector Machine Discriminant Analysis (SVMDA), and Support Vector Machine Regression (SVMR).

### SUMMARY OF THE INVENTION

The present invention is directed to a method and system for monitoring the biomass in algal bioreactors using LRRS.

According to one aspect of the presently disclosed subject matter, there is provided a method for monitoring invasive species in a bioreactor including:(a) providing a low resolution Raman spectrometer (LRRS) system and a digital computer configured for implementing signal processing algorithms; (b) an ex-situ method which includes the steps
(i) providing a known sample of a suspension including a biomass,
(ii) providing a calibration set of one or more known samples of a suspension, each known sample including a mixture of the biomass and a known concentration of at least one invasive species,
(iii) measuring LRRS spectra for the samples in steps (i) and (ii),
(iv) using a spectrum preprocessing algorithm to transform the LRRS spectra into normalized spectral data vectors,
(v) generating one or more Support Vector Machine (SVM) models for classifying the normalized spectral data vectors, and
(vi) determining an SVM support vector associated with each of the SVM models;
and (c) an in-situ method which includes the steps
(vii) providing an unknown sample of a suspension comprising a biomass,
(viii) measuring at least one LRRS spectrum for the unknown sample,
(ix) using the spectrum preprocessing algorithm to transform the at least one LRRS spectrum into at least one normalized spectral data vector, and
(x) using the one or more SVM models and the SVM support vectors to determine an output vector (Fs) containing one or more probability values corresponding to the presence of one or more invasive species in the unknown sample.

According to some aspects, the in-situ method provides an additional output vector (Fc) containing one or more probability values corresponding to one or more concentrations of the one or more invasive species in the unknown sample.

According to some aspects, the LRRS system includes a laser source which emits pulsed or continuous illumination.

According to some aspects, the LRRS system includes a spectrometer and/or a fiber-optic probe.

According to some aspects, the bioreactor is an open or semi-enclosed raceway, or a photo-bioreactor.

According to some aspects, the biomass includes an algal species.

According to some aspects, the biomass includes spirulina.

According to some aspects, the one or more invasive species includes an invasive algal species and/or a cyanobacterial species.

According to some aspects, the one or more SVM models includes Support Vector Machine Discriminant Analysis (SVMDA) and/or Support Vector Machine Regression (SVMR).

According to some aspects, the one or more SVM models incorporates a radial basis function kernel.

According to some aspects, the spectrum preprocessing algorithm includes differentiation of a Raman measurement vector with respect to a Raman frequency shift.

According to some aspects, the spectrum preprocessing algorithm includes an autoscale function and/or a logarithmic transformation.

According to another aspect of the presently disclosed subject matter, there is provided a system for monitoring invasive species in a bioreactor including: a sample of a suspension which includes a biomass, the sample being placed inside a dark chamber and illuminated by a laser source; a fiber-optic probe which collects light scattered by the sample; a dedicated spectrometer which measures a scattered light intensity over a Raman spectral range; and a digital computer configured to implement signal processing algorithms. The latter include one or more Support Vector Machine (SVM) models for determining an output vector (Fs) containing one or more probability values corresponding to the presence of one or more invasive species in the sample.

According to some aspects, the laser source emits pulsed or continuous illumination.

According to some aspects, the one or more SVM models determines an additional output vector (Fc) containing one or more probability values corresponding to a concentration of the one or more invasive species in the sample.

According to some aspects, the signal processing algorithms include a spectrum preprocessing algorithm which differentiates a Raman measurement vector with respect to a Raman frequency shift.

According to some aspects, the spectrum preprocessing algorithm includes an autoscale function and/or a logarithmic transformation.

According to some aspects, the one or more SVM models incorporates a radial basis function kernel.

According to some aspects, the biomass includes an algal species.

According to some aspects, the one or more invasive species includes an invasive algal species and/or a cyanobacterial species.

### BRIEF DESCRIPTION OF THE DRAWINGS

FIG. 1: A schematic of an exemplary LRRS system, according to the invention.

FIGS. 2A-2H: Graphs of exemplary LRRS spectra of single-species samples.

FIG. 3: A block diagram of an exemplary ex-situ method for generating SVMDA and SVMR models to identify and quantify a known species present in a bioreactor, according to the invention.

FIG. 4: A block diagram of an exemplary in-situ method which uses the SVMDA and SVMR models of FIG. 3 to identify and quantify one or more unknown species present in a bioreactor, according to the invention.

FIG. 5: A block diagram of an exemplary spectrum preprocessing algorithm.

FIG. 6: A conceptual drawing of an exemplary SVM model algorithm.

FIG. 7: An exemplary “confusion matrix” table for the SVMDA models of FIG. 3.

FIG. 8: An exemplary graph of concentration estimation by the SVMR models of FIG. 3.

### DETAILED DESCRIPTION OF THE INVENTION

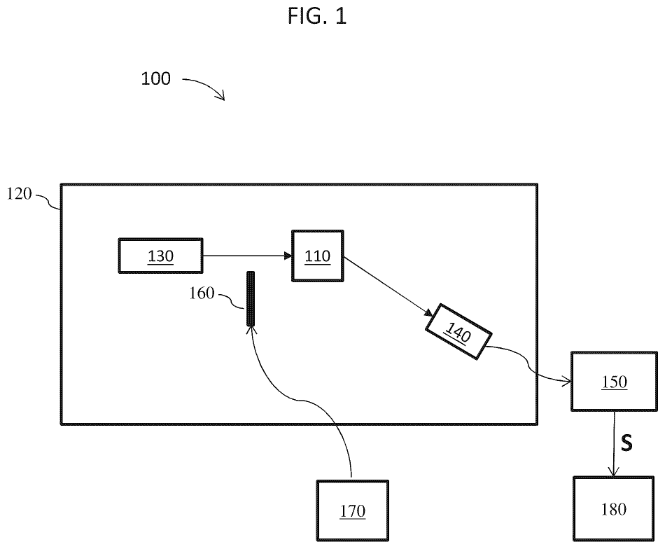

FIG. 1 shows a schematic of an exemplary LRRS system 100. A liquid sample 110 is placed inside a sample container having a typical volume of 300 μL (microliter). The container is positioned inside a dark chamber 120, at the focus of a laser source 130, which has, for example, a wavelength of 785 nm (nanometers) and an average power of typically several hundred milliwatts (mW). A fiber-optic probe 140, which collects light scattered by the sample, is connected to a dedicated spectrometer 150. The latter measures the scattered light intensity over a Raman spectral range, for example, of 160.7-4142.2 inverse centimeter (cm⁻¹), and with an exemplary spectral resolution of 6.0 cm⁻¹. The laser source may provide pulsed or continuous illumination of the sample. When using a continuous laser source, a shutter 160, under the control of a dedicated shutter controller 170, may be placed between the laser and the sample, as shown in FIG. 1, in order to facilitate pulsed excitation of the sample.

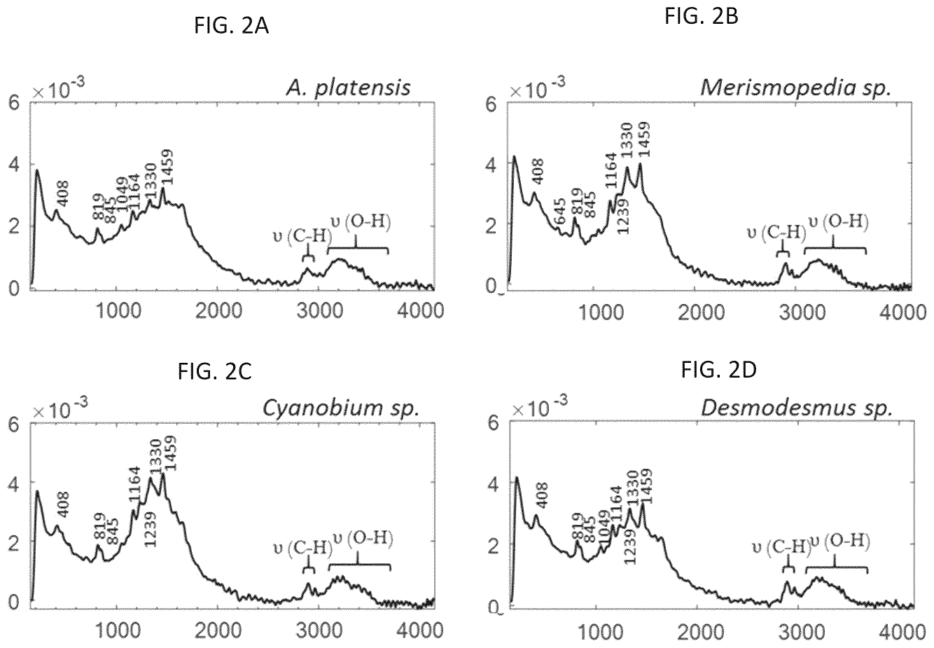

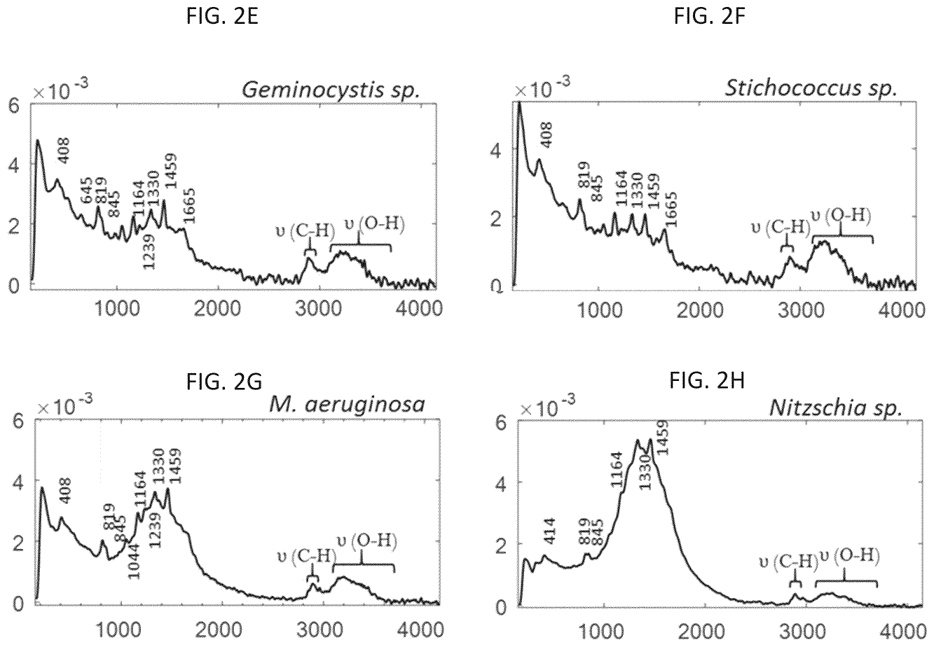

FIGS. 2A-2H shows graphs of exemplary LRRS spectra for a variety of single-species samples. Each sample consists of an aqueous suspension containing approximately 10⁴ cells/ml of a single species. In each figure, the Raman intensity, I(f), in arbitrary units is plotted on the vertical axis, and the Raman frequency shift (or wavenumber shift), f, in units of cm⁻¹ is plotted on the horizontal axis. Each spectrum is acquired by summing the intensities received in three consecutive two-second exposures.

FIG. 2A shows an LRRS spectrum for Arthrospira platensis, which is a variety of Spirulina. The spectra of cyanobacteria strains, such as Merismopedia sp., Cyanobium sp., Geminocystis sp., and Microcystis aeruginosa are shown in FIGS. 2B, 2C, 2E, and 2G, respectively. The spectra of algae strains, such as Desmodesmus sp., Stichococcus sp., and Nitzschia sp. are shown in FIGS. 2D, 2F, and 2H, respectively.

The different features in the spectra are due to the presence of numerous biomolecules at different concentrations within the cells. The major biomolecular information that is embedded within Raman spectra includes those of pigments, proteins, lipids, carbohydrates and nucleic acids. These biomolecules and pigments are detected as a result of the scattering caused by CH₃ bands, CH bending and CO, CC, CN stretching vibrations and OH vibrations, amongst others. The sensitivity and specificity of Raman spectroscopy are determined by the variations in composition and concentration of these molecules within the cells.

In each of the eight spectra shown, there is a wide peak in the region of 1000-1800 cm⁻¹, having several sub-peaks. The wide peak itself is a combination of the sub-peaks. The most intense peaks are observed between 400 and 1800 cm⁻¹, a region rich in structural information. The latter may be exploited to discriminate between the spectra of different species by using machine learning chemometric algorithms. Generally, the peak intensities are proportional to the species concentration.

To facilitate digital analysis, the intensity I(f) is converted into a Raman measurement vector S={I(fⱼ), j=1 to N}, where the sampled frequencies fⱼ are distributed uniformly on the horizontal axis and N is equal to the total number of sample points, which is typically greater than or equal to 1000. For example, in the spectra shown in FIGS. 2A-2H, N is equal to 1044. The Raman measurement vector S for each spectrum may be stored in a digital computer 180 for subsequent signal processing and analysis.

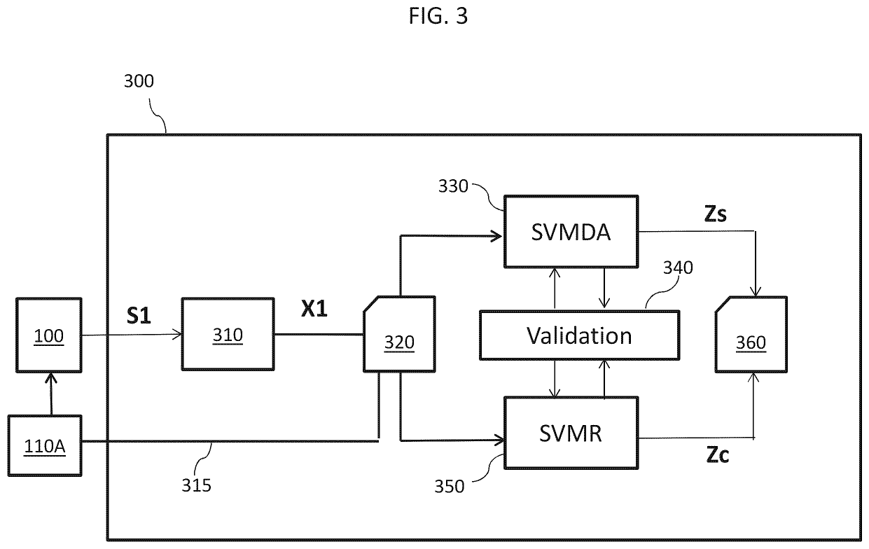

FIG. 3 shows a block diagram of an exemplary ex-situ method 300 for generating SVMDA and SVMR models to identify and quantify a known species present in a bioreactor, according to the invention. Block 110A provides a single-species sample containing a single species of known identity and known concentration. The species may be for example a single variant of Spirulina, cyanobacteria, or algae. LRRS system 100 generates a Raman spectrum measurement vector S1, which is transformed by spectrum preprocessing algorithm 310 into a normalized spectral data vector X1. The vector X1 is stored in spectral database 320, and the sampling loop 315 is repeated for additional samples provided by block 110A. In this way, a large collection of normalized spectral data vectors is stored in spectral database 320, for a set of calibration samples corresponding to different species and different concentrations.

SVMDA algorithm 330 generates support vectors Zs for determining the identity of the particular species present in the sample and SVMR algorithm 350 generates support vectors Zc for determining the concentration of the species. The support vectors are stored in SVM database 360. Validation module 340 uses statistical methods to cross-validate the data and to determine the selectivity and specificity of the SVM models, as explained further in the section below entitled Example.

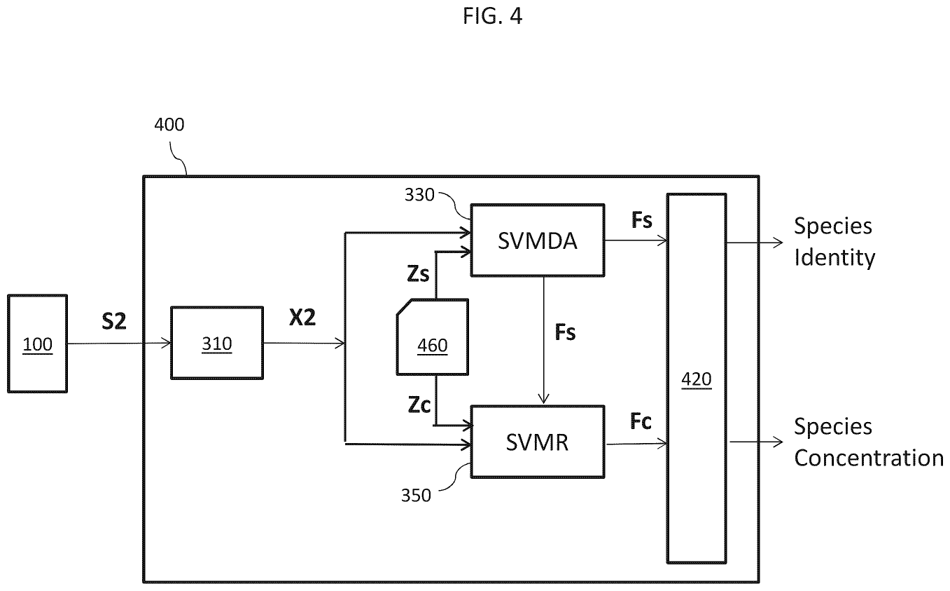

FIG. 4 shows a block diagram of an exemplary in-situ method 400 which uses the SVMDA and SVMR models and support vectors of FIG. 3 to identify and quantify one or more unknown species present in a bioreactor, according to the invention. LRRS system 100 generates a Raman spectrum measurement vector, which is transformed by spectrum preprocessing algorithm 310 into a normalized spectral data vector X2. The vector X2 is processed in the SVMDA and SVMR algorithms using the support vectors Zs and Zc, stored in the SVM database 460.

The ex-situ and in-situ methods of the invention, presented in FIGS. 3 and 4, respectively, may be implemented in the same digital computer, or in separate digital computers. In the former case, the SVM databases 360 and 460 may be one and the same. In the latter case, SVM database 460 is distinct from SVM database 360, and contains copies of the support vectors Zs and Zc that were previously determined in the ex-situ method and stored in SVM database 360.

The output vector Fs consists of values {Fsᵢ, i=1 to Ns}, where each value is a probability between 0 and 1 for the sample to belong to one of the Ns species modeled in the SVMDA algorithm block. Similarly, the output vector Fc consists of values {Fcⱼ, j=1 to N_{c}}, where each value is a probability between 0 and 1 for the sample to belong to one of the N_{c} species concentrations modeled in the SVMR algorithm block. Likelihood logic block 420 compares the probabilities in the output vectors with detection thresholds in order to determine which species, and what concentrations, are most likely to be present in the unknown sample.

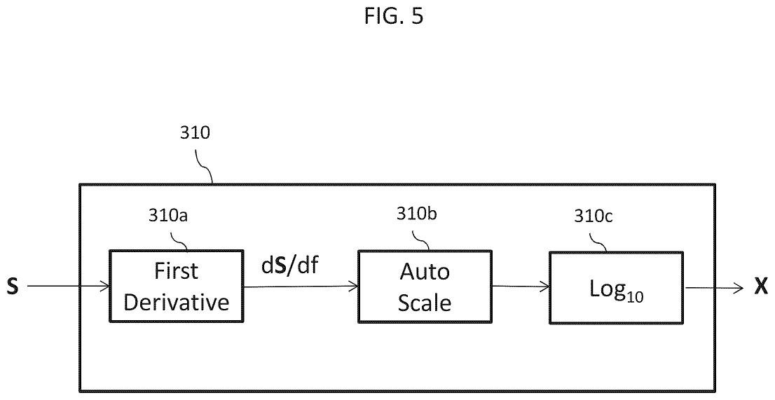

FIG. 5 shows a block diagram of the exemplary spectrum preprocessing algorithm 310. Block 310a generates a first derivative of the Raman measurement vector S, with respect to the Raman frequency shift, f. The resulting vector, denoted by dS/df, has the components:

$${( dS / df )}_{j} = ( {S}_{j + 1} - {S}_{j - 1} ) / ( {f}_{j + 1} - {f}_{j - 1} ) \qquad ( equation 1 )$$

where (j=2, 3, . . . (N−1)).

Block 310b applies an autoscale function to dS/df, as described in version 8.6 of the Matlab Eigenvector PLS toolbox of MATLAB R2018a, both of which are available from Mathworks Inc. After a logarithmic transformation in block 310c, the resulting normalized spectral data vector, X, has the components given by:

$${X}_{j} = {log}_{10} [ \vert dS / {df}_{j} \vert / ( \sum \vert dS / {df}_{i} \vert ) ] \qquad ( equation 2 )$$

where i, j=2, 3, . . . (N−1), and the summation is over all (i).

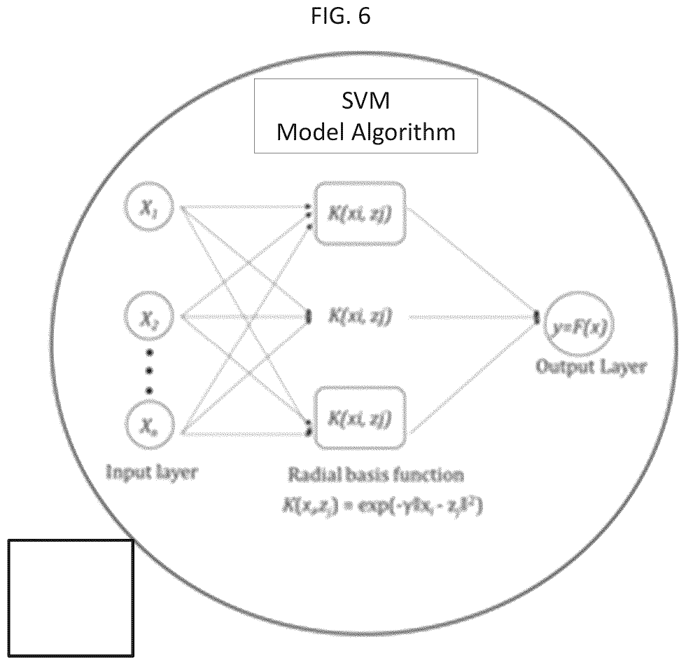

FIG. 6 shows a conceptual drawing of an exemplary SVM model algorithm. In SVMDA, a nonlinear, non-parametric method is used for classification modelling. Basically, a collection of spectral data vectors, such as those stored in spectral database 320, is separated into distinctive regions, or classes, defined by hyperplanes in a multi-dimensional space. For each class, the best separation is achieved by finding the hyperplane having the greatest distance to the nearest data point within the class and defining an associated support vector z. Various types of SVM kernel may be used to define the distance between an arbitrary data vector, x, and the support vector z. For example, a radial basis function (RBF) kernel is defined by:

$$K ( x , z ) = exp ( γ - {‖ x - z ‖}^{2} ) \qquad ( equation 3 )$$

The parameter γ is referred to as the width of RBF kernel, and ∥v∥² denotes the usual L2 norm, i.e. the sum of the squared components of a vector v.

### Example

SVM models are generated from a calibration set of samples using the ex-situ algorithm of FIG. 3, for each of the eight species represented in FIGS. 2A-2H. A testing set consisting of replicate samples of each of the eight species is processed by the ex-situ algorithm of FIG. 4, and the results are analyzed by various statistical measures.

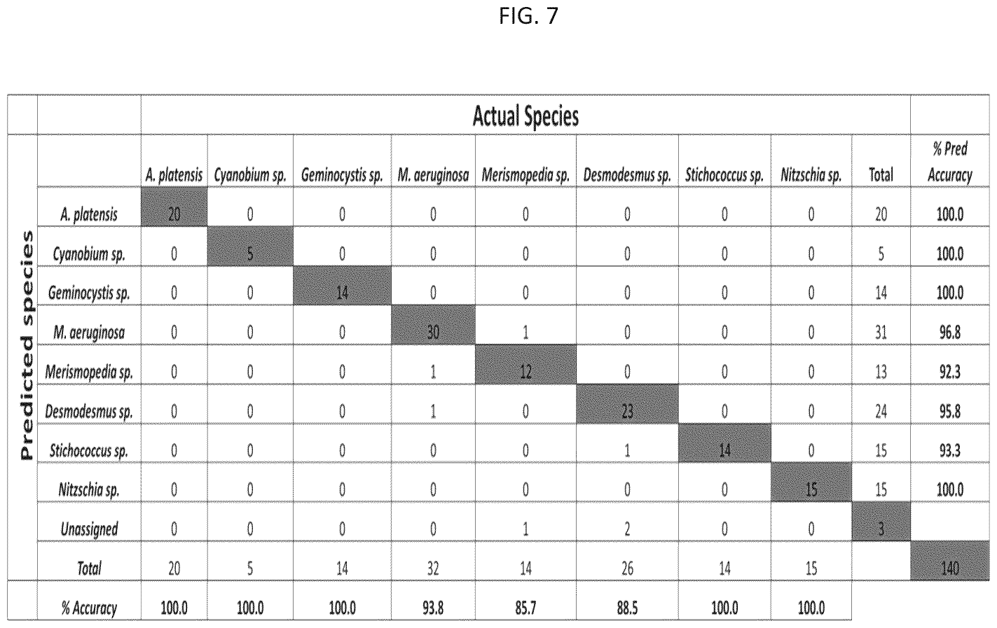

FIG. 7 shows an exemplary confusion matrix table obtained for the SVMDA models of FIG. 3. The tested samples consisted of 10⁶ cells/ml for each of the eight species listed. The prediction accuracy for species identification is shown in the last column. Of the 8 species tested, all had a prediction accuracy greater than 92%, and 4 of the species had a perfect prediction accuracy of 100%. Out of a total of 140 samples, 133 were correctly identified. The “unassigned” row indicates that 3 out of a total of 140 samples could not be identified as belonging to any of the eight species.

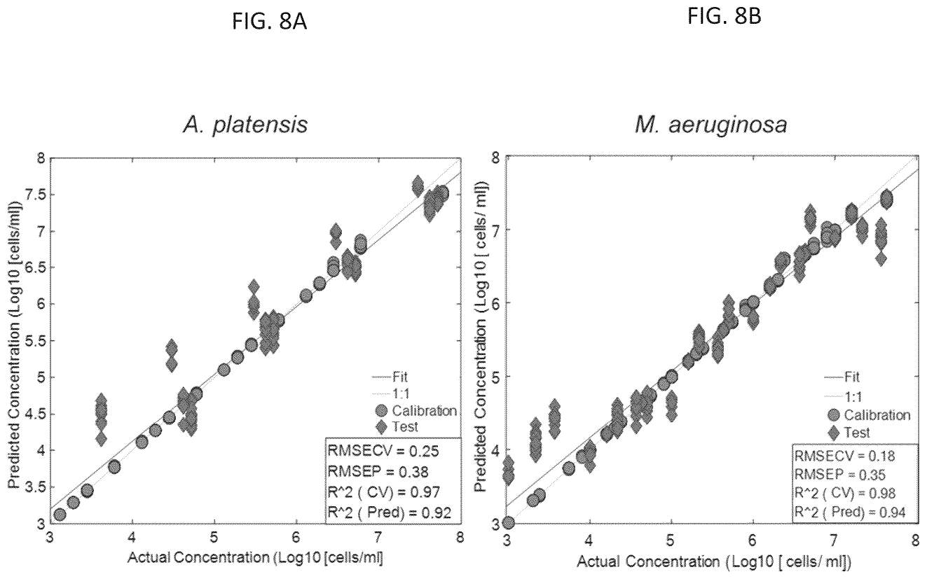

FIGS. 8A and 8B show exemplary graphs of concentration estimation by the SVMR models of FIG. 3, for two different species. In each graph, the logarithm to the base 10 of the predicted concentration is shown on the vertical axis, and that of the actual concentration is shown on the horizontal axis. The cross-validation samples used to calibrate the SVMR algorithm are shown by filled gray circles lying near the 1:1 line. The test samples, shown by filled red diamonds, are offset from the 1:1 line, and their scatter represents the observed errors in the predicted concentration. For the test samples, the coefficients of correlation (R²) between the predicted and actual concentrations are greater than or equal to 0.92 for both species, and the root-mean-square (RMS) values of the prediction error are less than or equal to 0.38 cells/ml for both species. This indicates the high reliability and accuracy of concentration estimation by the SVMR models of FIG. 3.

The descriptions of the various embodiments of the present disclosure have been presented for purposes of illustration, but are not intended to be exhaustive or limited to the embodiments disclosed. Many modifications and variations will be apparent to those of ordinary skill in the art of signal processing and machine learning algorithms without departing from the scope and spirit of the described embodiments. For example, other algorithms may be used in addition to or besides SVM algorithms.

The terminology used herein was chosen to best explain the principles of the embodiments, the practical application or technical improvement over technologies found in the marketplace, or to enable others of ordinary skill in the art to understand the embodiments disclosed herein.

1. A method for monitoring invasive species in a bioreactor comprising:
(a) providing a low resolution Raman spectrometer (LRRS) system and a digital computer configured for implementing signal processing algorithms;
(b) an ex-situ method further comprising steps
(i) providing a known sample of a suspension comprising a biomass,
(ii) providing a calibration set of one or more known samples of a suspension, each known sample comprising a mixture of the biomass and a known concentration of at least one invasive species,
(iii) measuring LRRS spectra for the samples in steps (i) and (ii),
(iv) using a spectrum preprocessing algorithm to transform the LRRS spectra into normalized spectral data vectors,
(v) generating one or more Support Vector Machine (SVM) models for classifying the normalized spectral data vectors, and
(vi) determining an SVM support vector associated with each of the one or more SVM models; and
(c) an in-situ method further comprising steps
(vii) providing an unknown sample of a suspension comprising a biomass,
(viii) measuring at least one LRRS spectrum for the unknown sample,
(ix) using the spectrum preprocessing algorithm to transform the at least one LRRS spectrum into at least one normalized spectral data vector, and
(x) using the one or more SVM models and the SVM support vectors to determine an output vector (Fs) containing one or more probability values corresponding to the presence of one or more invasive species in the unknown sample.

2. The method of claim 1 wherein the in-situ method provides an additional output vector (Fc) containing one of more probability values corresponding to concentrations of the one or more invasive species in the unknown sample.

3. The method of claim 1 wherein the LRRS system comprises a laser source which emits pulsed or continuous illumination.

4. The method of claim 1 wherein the LRRS system comprises a spectrometer and/or a fiber-optic probe.

5. The method of claim 1 wherein the bioreactor is an open or semi-enclosed raceway, or a photo-bioreactor.

6. The method of claim 1 wherein the biomass comprises an algal species.

7. The method of claim 1 wherein the biomass comprises spirulina.

8. The method of claim 1 wherein the one or more invasive species comprises an invasive algal species and/or a cyanobacterial species.

9. The method of claim 1 wherein the one or more SVM models comprises Support Vector Machine Discriminant Analysis (SVMDA) and/or Support Vector Machine Regression (SVMR).

10. The method of claim 1 wherein the one or more SVM models incorporates a radial basis function kernel.

11. The method of claim 1 wherein the spectrum preprocessing algorithm comprises differentiation of a Raman measurement vector with respect to a Raman frequency shift.

12. The method of claim 1 wherein the spectrum preprocessing algorithm comprises an autoscale function and/or a logarithmic transformation.

13. A system for monitoring invasive species in a bioreactor comprising:
a sample of a suspension comprising a biomass, the sample being placed inside
a dark chamber and illuminated by a laser source;
a fiber-optic probe which collects light scattered by the sample;
a dedicated spectrometer which measures a scattered light intensity over a
Raman spectral range; and
a digital computer configured to implement signal processing algorithms;
wherein,
the signal processing algorithms comprise one or more Support Vector Machine (SVM) models for determining an output vector (Fs) containing one or more probability values corresponding to the presence of one or more invasive species in the sample.

14. The system of claim 13 wherein the laser source emits pulsed or continuous illumination.

15. The system of claim 13 wherein the one or more SVM models determines an additional output vector (Fc) containing one or more probability values corresponding to concentrations of the one or more invasive species in the sample.

16. The system of claim 13 wherein the signal processing algorithms comprise a spectrum preprocessing algorithm which differentiates a Raman measurement vector with respect to a Raman frequency shift.

17. The system of claim 16 wherein the spectrum preprocessing algorithm comprises an autoscale function and/or a logarithmic transformation.

18. The system of claim 13 wherein the one or more SVM models incorporates a radial basis function kernel.

19. The system of claim 13 wherein the biomass comprises an algal species.

20. The system of claim 13 wherein the one or more invasive species comprises an invasive algal species and/or a cyanobacterial species.
